(19) United States
(12) Patent Application Publication
(10) Pub. No.: US 2024/0262876 A1
(43) Pub. Date: Aug. 8, 2024

(54) RECOMBINANT VACCINE AGAINST HELMINTHS IN PICHIA PASTORIS AND METHODS FOR PRODUCING AND PURIFYING PROTEINS FOR USE AS VACCINES AGAINST HELMINTHS

(71) Applicant: Fundacao Oswaldo Cruz, Rio de Janeiro (BR)

(72) Inventors: Miriam TENDLER, Rio de Janeiro (BR); Andrew J. SIMPSON, Sao Paulo (BR); Celso Raul Romero RAMOS, Rio de Janeiro (BR)

(73) Assignee: Fundacao Oswaldo Cruz, Rio de Janeiro (BR)

(21) Appl. No.: 18/443,739

(22) Filed: Feb. 16, 2024

Related U.S. Application Data
(62) Division of application No. 16/478,521, filed on Jul. 18, 2019, now abandoned.

(30) Foreign Application Priority Data
Jan. 23, 2017 (BR) 102017001309-0

(51) Int. Cl. C07K 14/435; A61K 39/00; C12N 15/81

(52) U.S. Cl. CPC C07K 14/43559; A61K 39/0003; C12N 15/815

(57) ABSTRACT

The present invention is related to the recombinant production of proteins by using a synthetic gene for high protein expression in Pichia pastoris. More specifically, the invention describes the production of Sm14 Schistosoma mansoni recombinant protein, where a synthetic gene was created to promote high expression of such protein, a gene which was cloned under control of two types of Pichia pastoris promoters: methanol-inducible promoter (AOXI) and constituent promoter (GAP). With these constructions, Pichia pastoris strains were genetically manipulated to efficiently produce vaccine antigen Sm14. The processes to produce and purify this protein from P. pastoris cells, which can be escalated for their industrial production, were also improved.

### CROSS-REFERENCE TO RELATED APPLICATIONS

This application is a divisional patent application of copending U.S. application Ser. No. 16/478,521, filed Jul. 18, 2019, which is a U.S. national stage application of international application number PCT/BR2018/000001, filed Jan. 22, 2018, which claims priority from Brazilian patent application number BR102017001309-0 filed on Jan. 23, 2017. The contents of each of these applications is hereby incorporated by reference.

### Incorporation by Reference of Material Submitted Electronically

The material contained in the Sequence Listing provided herewith in XML format in the file entitled “2024-02-15_Sequence_Listing_File_22053-00027.xml” created on Feb. 15, 2024 and containing 7989 bytes, is hereby incorporated by reference herein.

### FIELD OF APPLICATION

The present invention is related to the field of recombinant protein production using a synthetic gene associated with high protein expression in Pichia pastoris. More specifically, the invention describes the production of Sm14 Schistosoma mansoni recombinant protein, where a synthetic gene was created to promote high expression of such protein, a gene which was cloned under control of two types of Pichia pastoris promoters: methanol-inducible promoter (AOX1) and constituent promoter (GAP). With these constructions, Pichia pastoris strains were genetically manipulated to efficiently produce vaccine antigen Sm14. The processes to produce and purify this protein from P. pastoris cells, which can be escalated for their industrial production, were also improved.

### Invention Fundamentals

The Sm14 protein which has a molecular weight of approximately 14.8 kDa and belongs to the protein family which binds to fatty acids (Fatty Acid Binding Proteins, FABP), has already been widely studied and described by the applicant in their previous patent applications related to this technical matter.

The three-dimensional structure of protein Sm14 was predicted through molecular modeling by computerized homology, as well as crystallography and Nuclear Magnetic Resonance. The structure of protein Sm14 allowed us to identify potential protective epitopes and enabled us to use rSm14 as a vaccination antigen. This structure, as well as the models built for homologous Fasciola hepatica proteins (FABP type 3 being the one which shares greater sequential identity, 49%), shows that these molecules adopt three-dimensional configurations typical of the FABP family members). Protein Sm14 was the first FABP of parasites to be characterized. The scientific literature data indicate the parasites' FABPs as important targets for the development of drugs and vaccines against such infectious agents.

Therefore, based on the entire state-of-the-art of knowledge gathered by the inventors, it will be demonstrated here that protein Sm14 recombinant forms can provide high protection against infections caused by supposedly pathogenic helminths in relation to humans and animals.

In papers which demonstrated the protective activity of Sm14 for the first time, the corresponding recombinant protein was expressed with the pGEMEX-Sm14 vector in the form of inclusion bodies. After the bodies were isolated and washed, the protein was purified by preparative electrophoresis by electroeluting the corresponding band (Tendler et al., 1996). However, this methodology was not appropriate for producing proteins in a larger scale. Later, protein Sm14 started being produced with a fusion of six consecutive histidines (6×His) in the extreme amino terminal in Escherichia coli expression system, in the form of inclusion bodies. After the bodies were obtained and solubilized, refolding was required in order to obtain an immunologically active protein (Ramos et al., 2001).

The Brazilian patent application PI 1005855-9 (corresponding to U.S. Pat. Nos. 9,193,772 and 9,475,838) is related to the obtainment of a synthetic gene for protein Sm14 protein. The genetic transformation of Pichia pastoris with such synthetic gene under control of promoter AOX1 allows the production and purification of protein Sm14. The obtainment of protein Sm14 is reached from a synthetic gene, containing optimized codons for high Pichia pastoris expression.

However, it is noted that despite of the entire knowledge gained by the inventors, there are still disadvantages to be overcome to obtain an antigen material which can be obtained with high yield, in industrial scales under BPF conditions, and which does not lose the stability characteristics.

### SUMMARY OF THE INVENTION

This invention proposes a platform for producing a recombinant Sm14 vaccine antigen against helminths in Pichia pastoris. Through the referred platform, it is possible to obtain a recombinant vaccine against helminths (in P. pastoris), including the production and purification processes of protein Sm14 developed in the Pichia pastoris system.

The invention further proposes a synthetic gene for protein Sm14 expression. The genetic transformation of Pichia pastoris with such synthetic gene under control of the AOX1 and GAP promoters allows to produce and purify protein Sm14.

Thus, the invention allows to obtain protein Sm14 from a synthetic gene containing codons optimized for high expression in Pichia pastoris, SEQ ID NO:3 (final gene sequence), as well as protein Sm14 purification procedures.

### BRIEF DESCRIPTION OF THE FIGURES

FIG. 1 shows the strategy for building the pPIC9K-Sm14-MV and pGAP9K-Sm14-MV plasmids.

FIG. 2 shows the induction of protein Sm14 expression in P. pastoris GS115/pPIC9K-Sm14-MV.

FIG. 3 shows protein Sm14 expression in P. pastoris GSI15/pGAP9K-Sm14-MV.

FIG. 4 shows the result of the induction of protein Sm14 expression in P. pastoris GS115/pPIC9K-Sm14-MV culture in fermenter.

FIG. 5 shows the western blot analysis of purified protein of P. pastoris.

FIG. 6 shows the circular dichroism spectrum of P. pastoris purified protein Sm14.

### DETAILED DESCRIPTION OF THE INVENTION

The main purpose of the invention which is to produce a recombinant vaccine against helminths, can be achieved by producing recombinant proteins using a synthetic gene for high protein expression in Pichia pastoris. According to the invention a synthetic gene was created to promote high expression of protein Sm14, and with such gene the Pichia pastoris strain was obtained and genetically manipulated to effectively produce a vaccine. This invention further contemplates this protein production and purification processes from P. pastoris cells; which can be escalated for industrial production.

A Pichia pastoris is an efficient host to express and secrete heterologous proteins. The main promoter used for expression in this system is the strongly and firmly regulated promoter from the alcohol oxidase 1 gene (AOX1), inducible by methanol. However, this invention describes a system using an alternative promoter to prevent the use of methanol. The promoter of enzyme glyceraldehyde 3 phosphate dehydrogenase (pGAP) gene was used in the present invention for the constituent expression of the heterologous protein, once this system is more appropriate for large scale production because it eliminates the risk and costs associated with the storage and supply of large volume of methanol.

The invention will now be described through its best execution process. The technical matter of patent application PI 1005855-9 which reflects the closest state-of-the-art of the present invention will be attached here for reference purposes.

1. Obtainment of Pichia pastoris Recombinant Strain
1.1 Synthetic Gene for Sm14 Expression in P. pastoris:

First a gene was designed and synthesized containing optimized codons to obtain maximum expression of protein Sm14 in P. pastoris. In the present case protein Sm14-MV was used; however any form of protein Sm14 can be used.

There is evidence in the literature about the differential use of codons between proteins expressed in low amount and high amount, in the same organism (Roymondal and Sahoo, 2009). However, the codon use tables available in databases (for example: (www.kazusa.or.jp/codon) contain data from all body proteins, without taking the gene expression level into account. For this reason, in order the gene design, a codon use table was first elaborated based on data about sequences that codify recombinant proteins expressed over 1 gram per culture Liter in P. pastoris (see Table 1), as well as the sequence for AOX1 protein (which represents 30% of total P. pastoris protein, after induction with methanol). 1

TABLE 1
List of high-expression recombinant proteins in P. pastoris.

| Expressed | | |
|---|---|---|
| protein | (mg/L) | Reference |
| Hydroxynitrile | 22000 | Hasslacher, M. et al. (1997) Protein |
| lyase | | Expr. Purif. 11: 61-71 |
| Mouse gelatin | 14800 | Werten, M. W. et al. (1999) Yeast 15: |
| | | 1087-1096 |
| Tetanus toxin | 12000 | Clare, J. J. et al. (1991) Bio/Technology |
| fragment C | | 9: 455-460 |
| Human tumor | 10000 | Sreekrishna, K. et al. (1989) Biochemistry |
| necrosis factor | | 28: 4117-4125 |
| α-amylase | 2500 | Paifer, E. et al. (1994) Yeast 10: |
| | | 1415-1419 |
| T2A peroxidase | 2470 | Thomas, L. et al. (1998) Can. J. Microbiol. |
| | | 44: 364-372 |
| Catalase L | 2300 | Calera, J. A. et al. (1997) Infect. Immun. |
| | | 65: 4718-4724 |
| Hirudin | 1500 | Rosenfeld, S. A. et al. (1996) Protein |
| | | Expr. Purif. 8: 476-482. |

For gene design, protein Sm14-MV sequence, which has a valine residue at position 62 has been chosen—in place of cystein, which makes it more stable (Ramos et al., 2009); represented here as SEQ ID NO:1.

SEQ ID NO: 1
MSSFLGKWKL SESHNFDAVM SKLGVSWATR QIGNTVTPTV
TFTMDGDKMT MLTESTFKNL SVTFKFGEEF DEKTSDGRNV
KSVVEKNSES KLTQTQVDPK NTTVIVREVD GDTMKTTVTV
GDVTAIRNYK RLS

After the first selection of codons according to the table created with protein data from Table 1, the clearance of sequences which resulted in transcription termination sites (ATTTA), splicing cryptic donors and receptors (MAGGTRAGT and YYYNTAGC, respectively) and repetitive sequences (cut off sites for restriction enzymes BamHI and EcoRI) was conducted. SEQ ID NO:2 shows the sequence designed to express protein Sm14-MV in Pichia pastoris.

| SEQ ID NO: 2 | |
|---|---|
| 1 | ATGTCTTCTT TCTTGGGTAA GTGGAAGTTG TCTGAATCTC ACAACTTCGA |
| 51 | CGCTGTTATG TCTAAGTTGG GTGTTTCTTG GGCTACCAGA CAAATTGGTA |
| 101 | ACACCGTTAC TCCAACCGTT ACCTTCACCA TGGACGGTGA CAAGATGACT |
| 151 | ATGTTGACCG AGTCTACCTT CAAGAACTTG TCTGTTACTT TCAAGTTCGG |
| 201 | TGAAGAGTTC GACGAAAAGA CTTCTGACGG TAGAAACGTT AAGTCTGTTG |
| 251 | TTGAAAAGAA CTCTGAATCT AAGTTGACTC AAACTCAAGT TGACCCAAAG |
| 301 | AACACTACCG TTATCGTTAG AGAAGTTGAC GGTGACACTA TGAAGACTAC |
| 351 | TGTTACCGTT GGTGACGTTA CCGCTATCAG AAACTACAAG AGATTGTCTT |
| 401 | AA |

The Kozak sequence of protein AOX1 gene of P. pastoris (AAACG) was added to the 5′-end of the designed sequence. Finally, the restriction sites for BamHI (GGATCC) and EcoRI (GAATTC) were added to 5′ and 3′-ends of the designed gene, respectively.

SEQ ID NO:3 shows the final sequence of the synthetic gene for protein Sm14 production.

| SEQ ID NO: 3 | |
|---|---|
| 1 | GGATCCAAAC GATGTCTTCT TTCTTGGGTA AGTGGAAGTT GTCTGAATCT |
| 51 | CACAACTTCG ACGCTGTTAT GTCTAAGTTG GGTGTTTCTT GGGCTACCAG |
| 101 | ACAAATTGGT AACACCGTTA CTCCAACCGT TACCTTCACC ATGGACGGTG |
| 151 | ACAAGATGAC TATGTTGACC GAGTCTACCT TCAAGAACTT GTCTGTTACT |
| 201 | TTCAAGTTCG GTGAAGAGTT CGACGAAAAG ACTTCTGACG GTAGAAACGT |
| 251 | TAAGTCTGTT GTTGAAAAGA ACTCTGAATC TAAGTTGACT CAAACTCAAG |
| 301 | TTGACCCAAA GAACACTACC GTTATCGTTA GAGAAGTTGA CGGTGACACT |
| 351 | ATGAAGACTA CTGTTACCGT TGGTGACGTT ACCGCTATCA GAAACTACAA |
| 401 | GAGATTGTCT TAAGAATTC |

Following the synthesis of the designed sequence (SEQ ID NO:3), the cloning and later sequencing of the synthetic gene was performed in vector pCR2.1 to confirm the fidelity of the synthesized sequence with the designed sequence.

1.2 Constructions of Plasmids for Protein Sm14 Expression in Pichia pastoris:

The synthesized gene was cloned in vector pPIC9K where protein Sm14 is expressed without any fusion, allowing its intracellular production.

Vector pPIC9K (Invitrogen) was chosen for the construction of Sm14 expression plasmid in P. pastoris tor the following reasons:
(1) Possibility for use to express intracellular proteins replacing the alpha-factor gene with the gene of interest, through the vector's BamHI restriction site, located before the Kozak sequence and the beginning of translation. For this purpose, it was necessary to recreate the Kozak sequence before the ATG of the ORF to be expressed, according to Sm14's synthetic gene design.
(2) It has the advantage to allow the selection of clones with multiple copies integrated into the genome, by selecting resistance against antibiotic G418. This possibility did not exist with pPIC9, previously used.

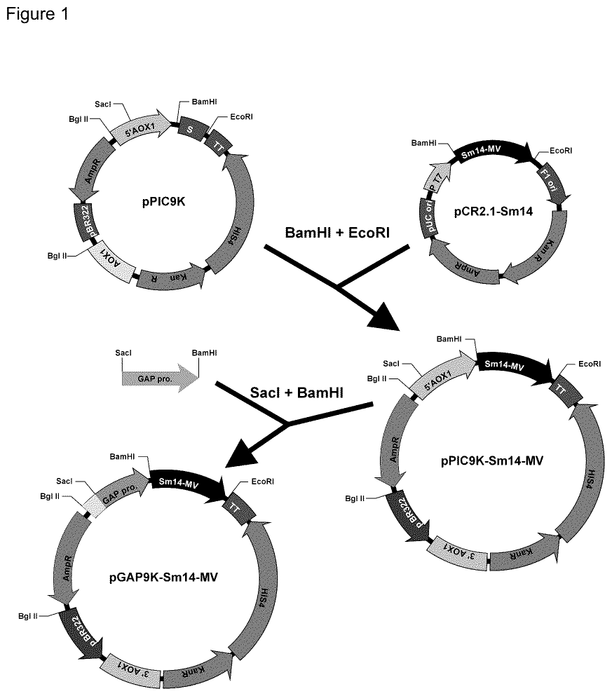

The strategy for building plasmid pPIC9K-Sm14 is described in FIG. 1. According to this strategy, plasmid pCR21-Sm14-MV and vector pPIC9K were digested with restriction enzymes BamHI and EcoRI. After digestion, the DNA fragments were separated by agarose gel electrophoresis and the fragments corresponding to vector pPIC9K and to the synthetic Sm14-MV insert were excised from the agarose gel, purified and bond using T4 DNA ligase. E. coli's DH5α strain was transformed by link reaction and the clones were selected in LB agar medium containing ampicillin. The pDNA of some ampicillin-resistant clones was purified and analyzed by restriction with enzymes BamHI and EcoRI, as well as by DNA sequencing. The construction so obtained was called pPIC9K-Sm14-MV, which expresses protein Sm14 under control of the strong alcohol oxidase 1 (AOX1) promoter, which is induced by methanol (Gregg et al., 1993).

According to the present invention, another promoter was successfully used in the expression of recombinant proteins in Pichia pastoris, the constituent promoter of glyceraldehyde-3-phosphate dehydrogenase (GAP) gene. For the large scale production of recombinant proteins, the promoter GAP-based expression system is more appropriate than promoter AOX1, because the risks and costs associated with the transport and storage of large volume of methanol are eliminates (Zhang et al, 2009).

According to the strategy described in FIG. 1, promoter GAP was amplified from the genomic DNA of strain GS115 of Pichia pastoris, using primers GAP-for (SEQ ID NO: 4) and GAP-rev (SEQ ID NO:5), which contain the restriction sites Sac and BamHI, respectively.

| SEQ ID NO: 4 | |
|---|---|
| GAGCTCTTTT TTGTAGAAAT GTCTTGG | 27 |
| SEQ ID NO: 5 | |
| GGATCCAAAT AGTTGTTCAA TTGATTGAAA TAGG | 34 |

With these oligonucleotides, a fragment of 506 pairs of bases corresponding to promoter GAP of Pichia pastoris was amplified (SEQ ID NO: 6).

| SEQ ID NO: 6 | |
|---|---|
| GAGCTCTTTT TTGTAGAAAT GTCTTGGTGT CCTCGTCCAA TCAGGTAGCC | 50 |
| ATCTCTGAAA TATCTGGCTC CGTTGCAACT CCGAACGACC TGCTGGCAAC | 100 |
| GTAAAATTCT CCGGGGTAAA ACTTAAATGT GGAGTAATGG AACCAGAAAC | 150 |
| GTCTCTTCCC TTCTCTCTCC TTCCACCGCC CGTTACCGTC CCTAGGAAAT | 200 |
| TTTACTCTGC TGGAGAGCTT CTTCTACGGC CCCCTTGCAG CAATGCTCTT | 250 |
| CCCAGCATTA CGTTGCGGGT AAAACGGAGG TCGTGTACCC GACCTAGCAG | 300 |
| CCCAGGGATG GAAAAGTCCC GGCCGTCGCT GGCAATAATA GCGGGCGGAC | 350 |
| GCATGTCATG AGATTATTGG AAACCACCAG AATCGAATAT AAAAGGCGAA | 400 |
| CACCTTTCCC AATTTTGGTT TCTCCTGACC CAAAGACTTT AAATTTAATT | 450 |
| TATTTGTCCC TATTTCAATC AATTGAACAA CTATTTTTGA ACAACTATTT | 500 |
| GGATCC | 506 |

The amplified promoter GAP's DNA, as well as plasmid pPIC9K-Sm14-MV was digested with restriction enzymes SacI and BamHI. The fragments corresponding to vector pPIC9K-SM14-MV and to insert GAP were purified from agarose gel, purified and bonded. E. coli's DH55a strain was transformed by link reaction and the clones were selected in LB agar medium containing ampicillin. The pDNA of some ampicillin-resistant clones was purified and analyzed by restriction with enzymes BamHI and EcoRI, as well as by DNA sequencing. The construction so obtained was called pGAP9K-Sm14-MV, which expresses the protein under control of constituent promoter pGAP.

1.3 Transformation of P. pastoris with Plasmids pPIC9K-Sm14-MV and pGAP9K-Sm14-MV. And Selection of Recombinant Clones with Multiple Copies

In order to produce the protein, the GS115 (his4) of P. pastoris strain was transformed with plasmids pPIC9K-Sm14-MV and pGAP9K-Sm14-MV, digested with enzymes SacI. With the DNA digested and purified, the competent cells for electroporation of strain GS115 were transformed.

After transformation, the cells were spread in RD medium (histidine-free medium, which contains: 1 M sorbitol; 2% dextrose; 1.34% YNB; 4×10−⁵% biotin; and 0.005% of each amino acid: L-glutamate, L-methionine, L-lysine, L-leucine and L-isoleucine for the selection of strains transformed by auxotrophy marker his4. Clones that managed to grow in the histidine-free medium were submitted to selection with antibiotic G418, at the concentrations of 0.5; 1; 2; and 4 mg/ml, in a YPD culture (1% Yeast Extract, 2% Peptone; 2% dextrose) at 30° C.

In order to confirm whether the selected clones presented the expression cassette of the synthetic gene, genomic DNA of the clones was purified and used in PCR reactions with primers AOX5′ and AOX3′ (for pPIC9K-Sm14-MV) and GAP5′ and AOX3′ (for pGAP9K-Sm14-MV).

### 1.4 Cultivation Expression in Shaker

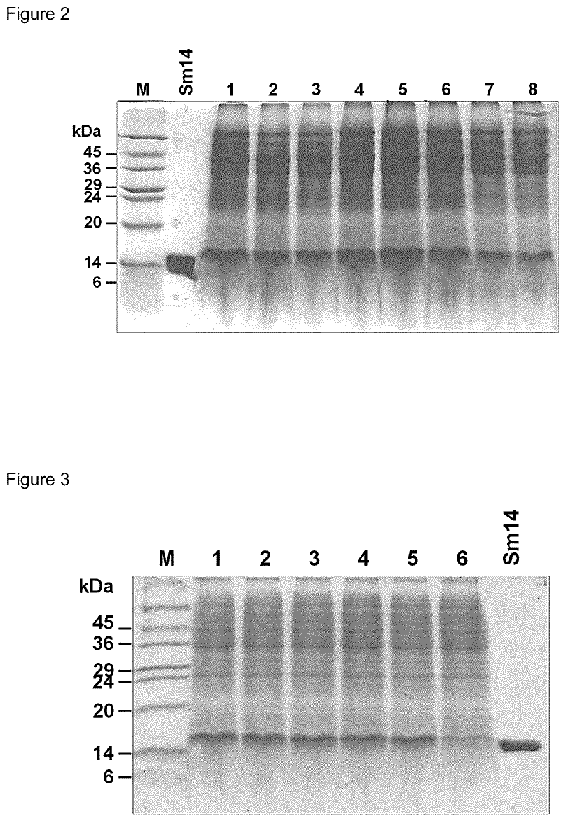

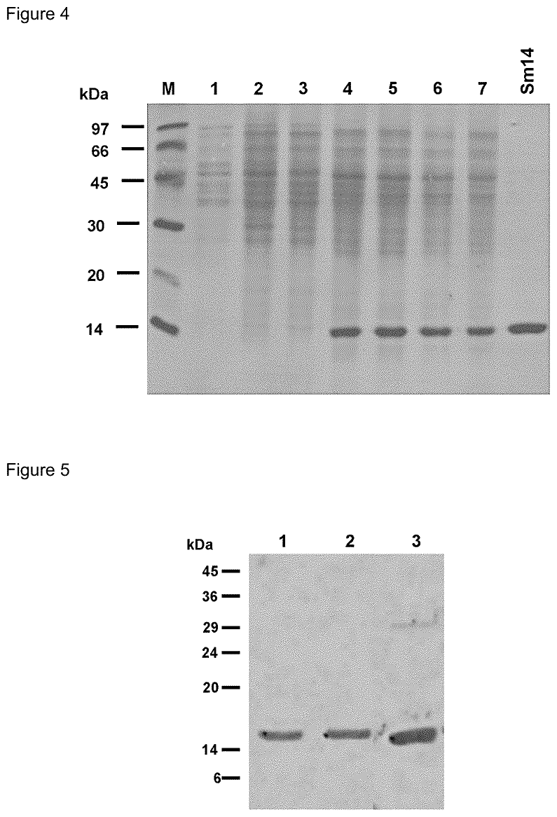

In order to test the expression of protein Sm14 in P. pastoris/pPIC9K-Sm14-MV clones, the clones were grew in shaker in 5 ml of the BMG medium (Buffered Minimal Glycerol medium, containing: 1.34% YNB; 0.04% biotin, 0.1 M potassium phosphate pH 6.0; and 1% glycerol) at 30° C. for 48 hours and then transferred to a BMM medium, 5 ml, (Buffered Minimal Methanol medium, containing the same components as the BMG medium, except for glycerol which was replaced with 0.5% methanol), for the induction of recombinant protein expression. After 72 hours, adding 0.5% methanol every 24 hours, the total proteins of each clone were analyzed by SOS-PAGE (FIG. 2). FIG. 2 shows the result of inducing the expression of protein Sm14 in P. pastoris clones GS115/pPIC9K-Sm14-MV.
M.—Low Molecular Weight Marker
Sm14.—Control of protein Sm14 without purified fusion of E. coli
1 to 8.—Total protein of clones 1-17 GS115/pPIC9K-Sm14-MV after induction with methanol.

In order to test the expression of protein Sm14 in P. pastoris/pGAP9K-Sm14-MV clones, the clones were grown in 5 ml of the BMG medium for only 72 hours. FIG. 3 shows the result of protein Sm14 expression in P. pastoris GS115/pGAP9K-Sm14-MV clones.
M.—Low Molecular Weight Marker
1 to 6.—Total protein of clones 1-17 GSI15/pGAP9KSm14-MV following 3 days of culture in BMG medium.
Sm14.—Control of protein Sm14 without purified fusion of E. coli

In all the selected clones, it was possible to note a majority band which coincides with the size of purified E. coli purified fusionless Sm14 protein.

1.5 Expression of Sm14 Recombinant in P. pastoris GS115/pPIC9K-Sm14-MV in Fermenter.

Fermentation was conducted with a 5-Liter culture in a fermenter with automatic supply, pH, antifoaming adjustments. The fermentation medium used was Basal Salt Medium (BSM) supplemented with the metal salt solutions (trace elements) and biotin.

In order to prepare the inoculum, an ampoule from the working bank of Pichia pastoris GS115/pPIC9K-Sm14-MV strain was used to inoculate the YDP medium and cultivated in shaker for 18 hours at 220 rpm, 30° C. Subsequently, the cells are inoculated in BSM medium supplemented with glycerol, cultivate in shaker for 18 hours before inoculating the fermenter.

The first fermentation phase (Glycerol fed batch) is started by adding 50% glycerol containing trace elements (12%) to the culture. The temperature and pH were maintained at 30° C. and 5.00, respectively and aeration in 1.0 VVM.

Induction was starting by adding 100% methanol, containing trace elements (12%) to the fermenter. Following the adaptation period, methanol was added according to the dissolved oxygen behavior.

FIG. 5 shows the induction of protein Sm14 expression in P. pastoris GS115/pPIC9K-Sm14-MV, in fermenter, where:
M.—Low Molecular Weight Marker
1-7.—Induction of expression for 24, 48, 72 and 96, 120, 144 and 168 hours, respectively.
Sm14.—Control of protein Sm14 without purified fusion of E. coli

Thus, the recombinant protein-specific induction corresponding to Sm14 in P. pastoris in the cultivation in fermentation was proven.

The yield of wet molecular mass reached of 170 g per liter and the estimated yield of Sm14 is of approximately 1 g of protein per culture liter. By changing the fermentation conditions, it is possible to exceed the cell mass yield and, correspondently the yield obtained of protein Sm14.

Even so, the obtained value already corresponds to a high protein Sm14 expression level, which comprises the majority protein of the cells following induction with methanol.

### 2. Purification of Recombinant Protein Sm14 Expressed in P. Pastoris GSI15/pPIC9K-Sm14-MV Strain

The purification protocol for recombinant protein Sm14 from P. pastoris cytoplasm was based on the methodology developed at the Experimental Schistosomiasis Laboratory of the Oswaldo Cruz Institute (IOC) for Sm14 purification without fusion into the E. coli system.

Lysis: The purification of recombinant protein starts with the lysis of P. pastoris cells. For that purpose, the cells are resuspended 30 mM Tris-HCl 30 mM pH 9.5 and lysed through pressure at 1500 Bar. The lysate is clarified by centrifugation.

Conditioning: The clarified lysate is submitted to tangential filtration in the 100 kDa membrane in order to remove high molecular weight proteins, followed by concentration and diafiltration in the 3 kDa membrane with buffer 30 mM Tris-HCl 30 mM pH 9.5 until conductivity corresponds to the buffer value.

Capture: Clarified lysate is loaded in resin Q-Sepharose XL (GE Healthcare) resin, balanced with buffer A (30 mM Tris-HCl pH 9.5). Then, the culture is washed with buffer A, and the protein is eluted with buffer B (30 mM Tris-HCl pH 8.0) where protein Sm14 elutes with a higher purity degree.

Polishing: The eluted protein of resin Q-sepharose XL presents few contaminant proteins. In order to separate such proteins from protein Sm14, the gel-filtration in resin Sephacryl S100 HR (GE Healthcare) was used, using PBS pH 7.4 as mobile phase.

4. Analysis of Recombinant Protein Sm14 Produced in Pichia pastoris

The recombinant protein was purified from P. pastoris by using the same physical-chemical characteristics as protein Sm14-MV without fusion expressed in E. coli. The protein so purified from P. pastoris corresponds in size with the protein specifically induced by methanol. Since we have the synthetic gene of protein Sm14-MV under control of AOX1 promoter in the expression cassette, we deduce that the expressed and purified P. pastoris protein is protein Sm14-MV.

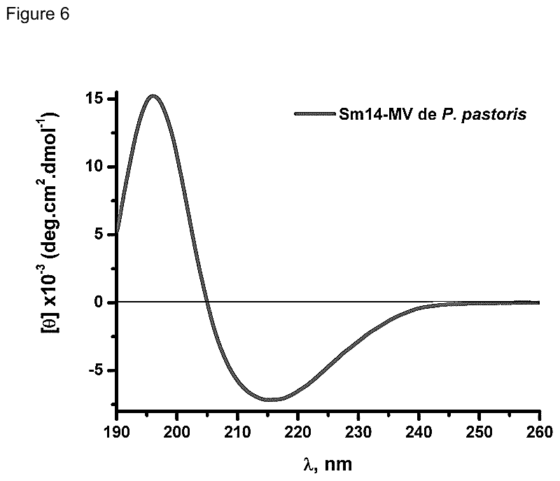

In order to confirm this statement, P. pastoris' purified protein was analyzed by western blot, by using rabbit anti-Sm14 serum (FIG. 6). FIG. 6 represents the western blot analysis of P. pastoris' purified protein.
1 and 2—Protein Sm14-MV purified from P. pastoris
3.—Positive control of protein Sm14 without purified fusion of E. coli

In this experiment, it was possible to observe that anti-Sm14 antibodies specifically recognized P. pastoris' purified protein (rabbit serum does not recognize endogenous P. pastoris proteins, data not shown), therefore confirming its identity.

Finally, it was necessary to identify whether the P. pastoris purified protein has a structure which corresponds to beta folding, characteristic of proteins of the Fatty Acid Binding Protein family, to which Sm14 belongs. To do so, protein samples were analyzed by circular dichroism, using spectral photopolarimeter J-815 (JASCO) (FIG. 7). FIG. 9 shows the circular dichroism spectrum of P. pastoris' purified protein Sm14.

As it can be noted in FIG. 7, the spectrum corresponds to a beta-structure protein. This spectrum was similar to circular dichroism spectra of lots of protein Sm14 previously purified from E. coli in laboratory.

Thus, based on the report above we may conclude that this invention allows us to:
design and synthesize a synthetic gene for high expression of protein Sm14-MV in Pichia pastoris;
build pPIC9K-Sm14-MV and pGAP9K-Sm14-MV expression plasmids containing the synthetic gene's sequence under control of AOX1 and GAP promoters, respectively;
obtain P. pastoris strains producing protein Sm14; and,
purify protein Sm14 in two chromatographic steps, feasible for scheduling for industrial production.

Through the process of the present invention, it was possible to build the pPIC9K-Sm14-MV and pGAP9K-Sm14MV plasmids for the constituent expression of protein Sm14 in Pichia pastoris and select the producing clones from the transformation of strain GS115 with plasmid pGAP9K-Sm14MV. One of the advantages of the invention is that the strains produce the protein within a lower time compared to the methanol induction system in constituent way. In addition, the new strain simplifies the fermentation process for the production of protein Sm14.

Therefore, the present invention described herein shows that protein Sm14 provides protection against Schistosoma mansoni infection in mice, in E. coli and P. pastoris platforms.

### REFERENCES

(1) Cregg, J. M., Vedvick, T. S. and Raschke, W. C. Recent advances in the expression of foreign genes in Pichia pastoris. BioTechnology v. 11, p. 905-910, 1993.
(2) Faber, K. N., Harder, W., and Veenhuis, M. Review: Methylotropic Yeasts as Factories for the Production of Foreign Proteins. Yeast. v. 11, p. 1331-1344, 1995.
(3) Ramos, C. R., Spisni, A., Oyama, S. Jr., Sforga, M. L., Ramos, H. R, Vilar, M. M., Alves, A. C., Figueredo, R. C., Tendler, M., Zanchin, N. I., Pertinhez, T. A., Ho, P. L. Stability improvement of the fatty acid binding protein Sm14 from S. mansoni by Cys replacement: structural and functional characterization of a vaccine candidate. Biochim Biophys Acta. v. 1794, p. 655-662, 2009.
(4) Ramos, C. R., Vilar, M. M., Nascimento, A. L., Ho, P. L., Thaumaturgo, N., Edelenyi, R., Almeida, M., Dias, W. O., Diogo, C. M., Tendler, M. r-Sm14—pRSETA efficacy in experimental animals. Mem Inst Oswaldo Cruz.v. 96, p. 131-135, 2001.
(5) Roymondal, U. D. and Sahoo, S. S. Predicting gene expression level from relative codon usage bias: an application to Escherichia coli genome. DNA Res. v. 16, p. 13-30, 2009.
(6) Tendler, M., Brito, C. A., Vilar, M. M., Serra-Freire, N., Diogo, C. M., Almeida, M. S., Delbem, A. C., Da Silva, J. F., Savino, W., Garratt, R. C., Katz, N., Simpson, A. S. A Schistosoma mansoni fatty acid-binding protein, Sm14, is the potential basis of a dual-purpose anti-helminth vaccine. Pree Natl Acad Sci USA. v. 93, p. 269-273, 1996.
(7) Zhang, A. L., Luo, J. X., Zhang, T. Y., Pan, Y. W., Tan, Y. H., Fu, C. Y., Tu, F. Z. Recent advances on the GAP promoter derived expression system of Pichia pastoris. Mol Biol Rep. v. 36, p. 1611-1619. 2009.

1. A method of purification of binding proteins of fatty acids, the method comprising the steps of:
(a) Performing a lysis of Pichia pastoris cells expressing a recombinant protein Sm14 of Schistosoma mansoni;
(b) clarifying the lysate obtained in stage (a) in order to obtain a clarified lysate;
(c) Conditioning the clarified lysate of step (b) through tangential filtration in preparation for performing ion exchange chromatography;
(d) loading the clarified lysate in an anion-exchange resin and after loading the protein, the protein is eluted by pH changes in the column;
(e) Separating the contaminant proteins from the recombinant protein Sm14 by gel-filtration, wherein the protein is coded by sequence SEQ ID NO: 3 and is expressed in Pichia pastoris; and
wherein the binding proteins of fatty acids is protein type-3 FABP of Fasciola hepatica.


SEQUENCE LISTING (6 sequences)

SEQ ID NO: 1 - protein, 133 aa, Schistosoma mansoni
MSSFLGKWKLSESHNFDAVMSKLGVSWATRQIGNTVTPTVTFTMDGDKMTMLTESTFKNLSVTFKFGEEFDEKTSDGRNVKSVVEKNSESKLTQTQVDPKNTTVIVREVDGDTMKTTVTVGDVTAIRNYKRLS

SEQ ID NO: 2 - DNA, 402 bp, Schistosoma mansoni
  source 1..402: mol_type = genomic DNA
atgtcttctttcttgggtaagtggaagttgtctgaatctcacaacttcgacgctgttatgtctaagttgggtgtttcttgggctaccagacaaattggtaacaccgttactccaaccgttaccttcaccatggacggtgacaagatgactatgttgaccgagtctaccttcaagaacttgtctgttactttcaagttcggtgaagagttcgacgaaaagacttctgacggtagaaacgttaagtctgttgttgaaaagaactctgaatctaagttgactcaaactcaagttgacccaaagaacactaccgttatcgttagagaagttgacggtgacactatgaagactactgttaccgttggtgacgttaccgctatcagaaactacaagagattgtcttaa

SEQ ID NO: 3 - DNA, 505 bp, Schistosoma mansoni
  source 1..505: mol_type = other DNA
gagctcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttttgaacaactatttggatcc

SEQ ID NO: 4 - DNA, 27 bp, Pichia pastoris
  source 1..27: mol_type = other DNA
gagctcttttttgtagaaatgtcttgg

SEQ ID NO: 5 - DNA, 34 bp, Pichia pastoris
  source 1..34: mol_type = other DNA
ggatccaaatagttgttcaattgattgaaatagg

SEQ ID NO: 6 - DNA, 506 bp, Schistosoma mansoni
  source 1..506: mol_type = other DNA
gagctcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttttgaacaactatttggatcc